(19) United States
(12) Patent Application Publication
(10) Pub. No.: US 2026/0041291 A1
(43) Pub. Date: Feb. 12, 2026

(54) TOILET SEAT WITH AI-ENABLED DIAGNOSTICS

(71) Applicant: Keith Ly, Pasadena, CA (US)

(72) Inventor: Keith Ly, Pasadena, CA (US)

(21) Appl. No.: 18/800,056

(22) Filed: Aug. 10, 2024

(51) Int. Cl. A47K 13/10; G01N 33/493

(52) U.S. Cl. CPC A47K 13/10; G01N 33/493

(57) ABSTRACT

According to an aspect of the present invention, there is provided a toilet seat comprising: multiple sensors for skin contacts with temperature; pressure sensors to asses body movements; a toilet seat base; one or more weight sensors in the toilet seat base to assess and monitor weight; and one or more lenses in the toilet seat base, wherein the lenses are configured to obtain an image of excrement in the toilet bowl.

### BACKGROUND

A number of prior art inventions relate to assessment by means of instruments within a toilet seat.

For example, U.S. Pat. No. 11,311,156 discloses a toilet seat with handles and light transmitters and light receivers. The toilet seat is positioned on a toilet, and the handles may be attached to the toilet seat such that a user can use them to lower and/or raise himself or herself onto/from the toilet seat. The light transmitters and receivers may be then used to determine blood oxygen saturation levels of the user, heart rate of the user, etc. The handles serve multiple purposes such as allowing a user to raise and lower himself or herself from a toilet and the handles provide a health monitoring function of determining blood oxygen saturation level, heart rate, diastolic pressure, and systolic pressure.

US7437781 discloses a toilet seat cover scale device for weighing a person or an object which includes, in combination, a toilet seat cover including hinges for hinged attachment to a toilet seat, a scale mechanism connected to the toilet seat cover, the scale mechanism having at least two force sensors located on a bottom of the toilet seat cover for contact with a toilet seat when the toilet seat cover is attached to a toilet seat and is in a closed position. The force sensors transmit the weight force measurements to a processor, which converts the weight force measurements to an accurate total weight measurement, and the processor transmits the accurate total weight measurement to a display.

Nevertheless, there is ample room from improvement of prior art toilet seats with regard to the range and efficacy of diagnostics.

### SUMMARY OF INVENTION

Therefore, the present invention provides a toilet seat with AI-enabled diagnostic and health monitoring capabilities conveniently utilizing images of excreta.

According to an aspect of the present invention, there is provided a toilet seat comprising: multiple sensors for skin contacts with temperature; pressure sensors to asses body movements; a toilet seat base; one or more weight sensors in the toilet seat base to assess and monitor weight; and one or more lenses in the toilet seat base, wherein the lenses are configured to obtain an image of excrement in the toilet bowl.

### BRIEF DESCRIPTION OF THE DRAWINGS

FIG. 1 illustrates a drawing of the AI-enabled health monitoring toilet seat according an embodiment.

FIG. 2 illustrates another view of the AI-enabled health monitoring toilet seat according an embodiment.

FIG. 3 illustrates another view of the AI-enabled health monitoring toilet seat according an embodiment.

FIG. 4 illustrates another view of the AI-enabled health monitoring toilet seat according an embodiment.

### DETAILED DESCRIPTION

The present invention allows persons to simply use the bathroom and take a picture of what is in the toilet bowel before they add toilet paper to send to an artificial intelligence machine learning server.

The AI-ML system can evaluate the color of the urine for example and provide some insights, conclusions, and/or recommendations to the user utilizing the toilet seat of the present invention.

The AI-ML system can differentiate between stool and urine and make appropriate adjustments to the analysis based on the presence of a mixture of stool and urine.

The AI-ML system of the present invention can calculates the degree of constipation or diarrhea, the volume of stool, and other elements. The system can analyze images for visual characteristics including consistency, fragmentation, edge fuzziness, and volume of stool.

Embodiments of the present invention will be described with reference to the attached drawings.

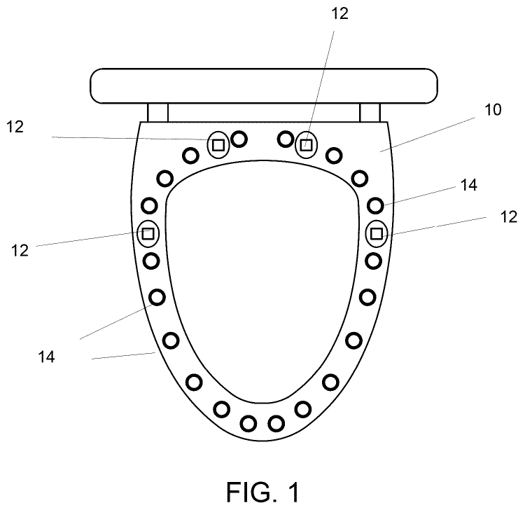

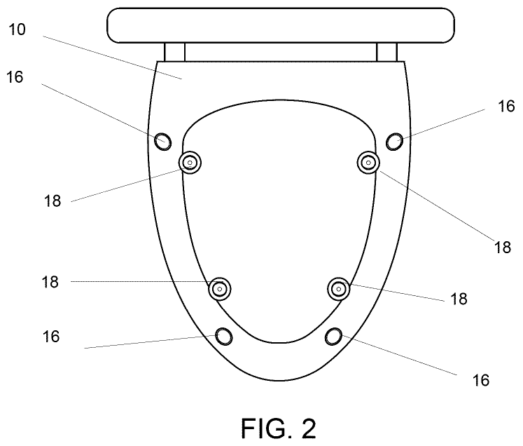

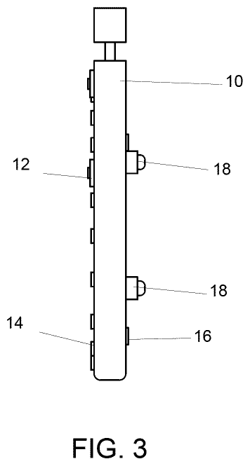

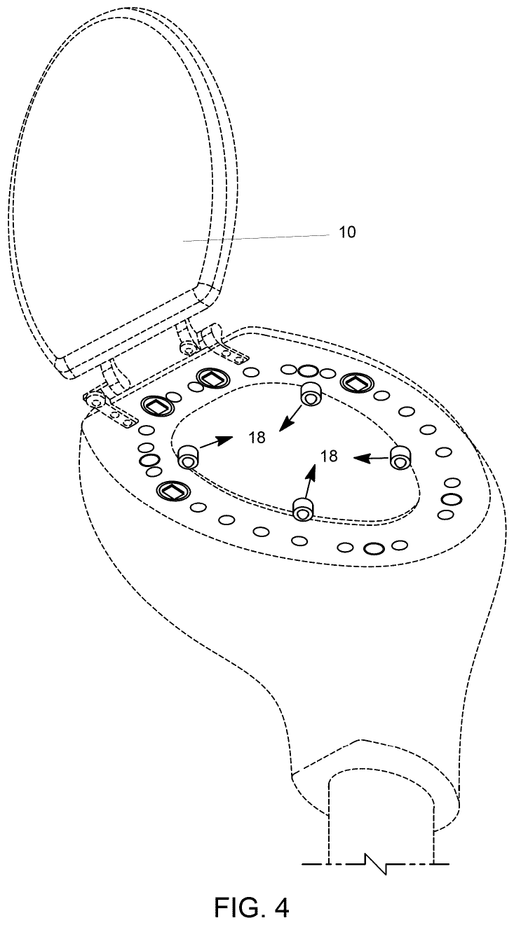

FIG. 1, FIG. 2, FIG. 3, and FIG. 4 illustrate in various views the AI-enabled health monitoring toilet seat according an embodiment.

Toilet seat 10 is an AI-enabled health monitoring toilet seat. Toilet seat 10 may have one or more lenses embedded somewhere.

Temperature Sensors 12 Measure Body Temperature Using Skin Contact with the user.

Pressure sensors 14 measure forces as a user sits down and gets up to assess balance, muscle tone, leg strength, and body power. Data gathered can also compare changes over time relative to base line.

Weight sensors 16 measure the body weight of a user and can be used to diagnose obesity.

Camera lenses 18 capture images of a bowl movement, urine sample, or combined sample. The lenses of embodiments of the present invention can be placed in a number of different locations in the toilet seat and can be, for example, circular, dome-shaped wide angle lenses, telescoping lenses, and/or lenses which are periscoping or otherwise inclined or use an instrument containing mirrors to get a view that would otherwise be blocked to be in direct line of sight with the toilet bowl.

The operation of the AI-enabled health monitoring toilet seat according an embodiment comprises:

First capturing the image of the bowl movement, urine sample, or combined sample by the lenses in the toilet seat of an embodiment of the present invention.

Then transmitting the image to an AI/ML server. The image can be transmitted by a communications module on the toilet seat of the present invention or may be electronically stored on a computer-readable storage medium associated with the toilet seat.

Then analyzing the image of the present sample by the AI/ML server. The AI/ML server has been trained by a training set of bowl movements along with data points corresponding to them.

Then outputting a report with health observations or recommendations. The observations and recommendations can be based on the appearance of stool, for example. Images of a person's poop can indicate if the person eating enough fiber and drinking enough water, or if their digestive system is processing food too slowly or too quickly.

Also, lasting changes in bowel habits or the appearance of a person's poop can be a sign of a medical condition that requires treatment, and thus, the AI-ML system of the present invention can track the appearance of stool over time and flag potential warning signs of colorectal cancer among other serious gastrointestinal health conditions.

The observations and recommendations can be based on the appearance of urine. Hydration status is prominently indicated by the intensity of the color of urine from nearly colorless to dark yellow. An unusual urine color also can be a sign of a health problem. For instance, some urinary tract infections can turn urine milky white. Kidney stones, some cancers and other diseases sometimes make urine look red due to blood.

The AI-ML system can apply an algorithm which makes it possible for the system to adjust for the noise caused by urine in a stool analysis and vice-versa.

The present invention allows persons to simply use the bathroom daily and take a picture of what is in the toilet bowel before they add toilet paper to send to an artificial intelligence machine learning server to assess daily wellness charted overtime to assess pattern of health status quo or early changes thus pickup early signs of disease state such as dehydration, hypovolemia, anemia, etc. It is also a monitoring device for post hospitalization monitor and physical bodily assessment of health via pattern motions and get-up-and go early pattern of weakness.

The AI-ML system can evaluate the color of the urine for example and provide some insights, conclusions, and/or recommendations to the user utilizing the toilet seat of the present invention.

The AI-ML system can differentiate between stool and urine and make appropriate adjustments to the analysis based on the presence of a mixture of stool and urine.

The AI-ML system of the present invention can calculates the degree of constipation or diarrhea, the volume of stool, and other elements. The system can analyze images for visual characteristics including consistency, fragmentation, edge fuzziness, and volume of stool and will alert the physician of possible impending dehydration or change in physical health with related vitals signs.

The observations and recommendations can be based on the appearance of stool. Images of a person's poop can indicate if the eating enough fiber and drinking enough water, or if their digestive system is processing food too slowly or too quickly.

Also, lasting changes in your bowel habits or the appearance of a person's poop can be a sign of a medical condition that requires treatment, and thus, the AI-ML system of the present invention can track the appearance of stool over time and flag potential warning signs of colorectal cancer among other serious gastrointestinal health conditions.

The observations and recommendations can be based on the appearance of urine. Hydration status is prominently indicated by the intensity of the color of urine from nearly colorless to dark yellow. An unusual urine color also can be a sign of a health problem. For instance, some urinary tract infections can turn urine milky white. Kidney stones, some cancers and other diseases sometimes make urine look red due to blood.

The AI-ML system can apply an algorithm which makes it possible for the system to adjust for the noise caused by urine in a stool analysis and vice-versa.

The illustrations of embodiments described herein are intended to provide a general understanding of the structure of various embodiments, and they are not intended to serve as a complete description of all the elements and features of apparatus and systems that might make use of the structures described herein. Many other embodiments will be apparent to those of skill in the art upon reviewing the above description. Other embodiments may be utilized and derived therefrom, such that structural and logical substitutions and changes may be made without departing from the scope of this disclosure.

Figures are also merely representational and may not be drawn to scale. Similar numerals designate similar elements among the several figures. Certain proportions thereof may be exaggerated, while others may be minimized. Accordingly, the specification and drawings are to be regarded in an illustrative rather than a restrictive sense. Thus, although specific embodiments have been illustrated and described herein, it should be appreciated that any arrangement calculated to achieve the same purpose may be substituted for the specific embodiments shown.

This disclosure is intended to cover any and all adaptations or variations of various embodiments. Combinations of the above embodiments, and other embodiments not specifically described herein, will be apparent to those of skill in the art upon reviewing the above description. Therefore, it is intended that the disclosure not be limited to the particular embodiment(s) disclosed.

What is claimed is:

1. A toilet seat comprising:
multiple temperature sensors for skin contacts;
pressure sensors to assess body movements;
a toilet seat base;
one or more weight sensors in the toilet seat base to assess and monitor weight; and
one or more lenses in the toilet seat base, wherein the lenses are configured to obtain an image of excrement in the toilet bowl.

2. The toilet seat of claim 1, further comprising an electronic communications network access device.

3. The toilet seat of claim 2, further comprising a server connected via the electronic communications network access device.

4. The toilet seat of claim 3, wherein the server is configured for operations comprising:
processing an image of excrement in the toilet bowl using an artificial intelligence machine learning system;
providing an analysis of one or more of stool and urine characteristics.

5. The toilet seat of claim 4, wherein server makes adjustments to a stool analysis based on the presence of a mixture of stool and urine in additional to the physical body's contact data's from toilet seat sensors.

6. The toilet seat of claim 4, wherein server makes adjustments to a urine analysis based on the presence of a mixture of stool and urine.

7. The toilet seat of claim 1, further comprising a toilet seat back.
